(19) United States
(12) Patent Application Publication
(10) Pub. No.: US 2015/0351661 A1
(43) Pub. Date: Dec. 10, 2015

(54) NAVIGATED INDUCTION DETECTOR

(71) Applicant: BRAINLAB AG, Feldkirchen (DE)

(72) Inventors: Uli MEZGER, Heimstetten (DE); Rainer BIRKENBACH, Erding (DE)

(73) Assignee: BRAINLAB AG, Feldkirchen (DE)

(21) Appl. No.: 14/760,515

(22) PCT Filed: Jan. 18, 2013
(86) PCT No.: PCT/EP2013/050872

§ 371 Date: Jul. 13, 2015

(51) Int. Cl. A61B 5/06; A61B 5/00; A61B 19/00; A61B 5/0478

(52) U.S. Cl. CPC A61B 5/062; A61B 5/0478; A61B 5/7246; A61B 19/5244; A61B 5/7405; A61B 5/742; A61B 2560/0475

(57) ABSTRACT

A method of determining the position of an induction-susceptive device inside an anatomical body part, comprises the following steps: a) emitting, towards the anatomical body part and from an electromagnetic emitter device, an oscillating electromagnetic field having a predetermined first field strength; b) acquiring, by using an electromagnetic detector device, an electromagnetic detection signal describing a second field strength which corresponds to the field strength of the electromagnetic field in the proximity of the anatomical body part; c) determining, based on comparing the first field strength and the second field strength, whether the second field strength differs from the first field strength, and d) if the second field strength differs from the first field strength, issuing a corresponding position indicating signal from an indication device, which position indicating signal indicates the presence of the induction-susceptible device.

The present invention is directed to a method of determining the position of an induction-susceptible device inside an anatomical body part, a corresponding computer program and a corresponding program storage medium storing that computer program.

In medical procedures carried out on the human brain, such as tumour therapy carried out on a patient suffering from a brain tumour, it is often necessary to be aware of the presence and correct functioning of different functional areas of the brain. To this end, intra operative neurophysiologic monitoring of nerve functionality is presently used as the gold standard for the preservation of brain functions during neurosurgical interventions. Such a monitoring is carried out in particular by placement of epidural or subdural strip electrodes to a burr whole or under the edge of a craniotomy in order to detect electric signals for acquiring an electroencephalography (EEG).

However, the exact localization of such electrodes on the patient's brain remains uncertain for the surgeon during insertion of the electrode. Proper positioning of subdural electrodes through a burr whole is rather challenging for a surgeon. Presently, inserting the electrodes is a blind method because the real position of the electrode contacts over the gyri of the brain can only be visualised postoperatively with medical resonance imaging or computer tomography. If an error in the electrode position is then detected, usually several adjustments are required to find the optimal position of the electrode for nerve response assessment. Furthermore, proper electrode placement is also important when recording epileptic activity of a patient's brain in an attempt to define the epileptic focus. Mispositioning of the electrodes could lead to misconception of the origin of epilepsy. This may then require relocation of the electrodes which is associated with a further operation and therefore a significantly higher risk of infection.

The present invention thus seeks to provide a method and corresponding computer programme for online determination of the electrode position, i.e. for determining the position in particular while the electrode is being inserted.

This problem is solved by the subject-matter of any appended independent claim. Advantages, advantageous features, advantageous embodiments and advantageous aspects of the present invention are disclosed in the following and contained in the subject-matter of the dependent claims. Different advantageous features can be combined in accordance with the invention as long as technically sensible and feasible. In particular, a feature of one embodiment which has the same or similar function of another feature of another embodiment can be exchanged. In particular, a feature of one embodiment which supplements a further function to another embodiment can be added to the other embodiment.

It is the function of a marker to be detected by a marker detection device (for example, a camera or an ultrasound receiver or analytical devices, like CT or MRI), such that its spatial position (i.e. its spatial location and/or alignment) can be ascertained. The detection device is in particular part of a navigation system. The markers can be active markers. An active marker can for example emit electromagnetic radiation and/or waves, wherein said radiation can be in the infrared, visible and/or ultraviolet spectral range. The marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can also, however, exhibit a cornered—for example, cubic—shape.

A marker device can for example be a reference star or a pointer or one marker or more than one (individual) markers which are preferably in a predetermined spatial relationship. A marker device comprises one, two, three or more markers which are in case of two or more markers in a predetermined spatial relationship. This predetermined spatial relationship is in particular known to a navigation system and for example stored in a computer of the navigation system.

A “reference star” refers to a device with a number of markers, advantageously three markers, attached to it, wherein the markers are (in particular detachably) attached to the reference star such that they are stationary, thus providing a known (and advantageously fixed) position of the markers relative to each other. The position of the markers relative to each other can be individually different for each reference star used within the framework of a surgical navigation method, in order to enable the corresponding reference star to be identified by a surgical navigation system on the basis of the position of the markers relative to each other. It is therefore also then possible for the objects (for example, instruments and/or parts of a body) to which the reference star is attached to be identified and/or differentiated. In a surgical navigation method, the reference star serves to attach a plurality of markers to an object (for example, a bone or a medical instrument) in order to be able to detect the position of the object (i.e. its spatial location and/or alignment). Such a reference star in particular comprises a way of being attached to the object (for example, a clamp and/or a thread) and/or a holding element which ensures a distance between the markers and the object (in particular in order to assist the visibility of the markers to a marker detection device) and/or marker holders which are mechanically connected to the holding element and which the markers can be attached to.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements and optionally a volatile memory (in particular, a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or vibration element incorporated into an instrument

The method in accordance with the invention is in particular a data processing method. The data processing method is preferably performed using technical means, in particular a computer. The data processing method is in particular executed by or on the computer. The computer in particular comprises a processor and a memory in order to process the data, in particular electronically and/or optically. The calculating steps described are in particular performed by a computer. Determining steps or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term “computer” includes a cloud computer, in particular a cloud server. The term “cloud computer” includes a cloud computer system which in particular comprises a system of at least one cloud computer and in particular a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing” which describes computation, software, data access and storage services which do not require the end user to know the physical location and/or configuration of the computer delivering a specific service. In particular, the term “cloud” is used as a metaphor for the interne (world wide web). In particular, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer in particular comprises interfaces in order to receive or output data and/or perform an analogue-to-digital conversion. The data are in particular data which represent physical properties and/or are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals in particular represent the data received or outputted by the computer.

Preferably, the inventive method is at least partly executed by a computer. That is, all steps or just some of the steps (i.e. less than a total number of steps) of the inventive method may be executed by a computer.

The expression “acquiring data” encompasses in particular (within the framework of a data processing method) the scenario in which the data are determined by the data processing method or program. Determining data in particular encompasses measuring physical quantities and transforming the measured values into in particular digital data and/or computing the data by means of a computer, in particular computing the data within the method of the invention. The meaning of “acquiring data” in particular also encompasses the scenario in which the data are received or retrieved by the data processing method or program, for example from another program, a previous method step or a data storage medium, in particular for further processing by the data processing method or program. Thus, “acquiring data” can also for example mean waiting to receive data and/or receiving the data. The received data can for example be inputted via an interface. “Acquiring data” can also mean that the data processing method or program performs steps in order to (actively) receive or retrieve the data from a data source, for instance a data storage medium (such as for example a ROM, RAM, database, hard disc, etc.) or via the interface (for instance, from another computer or a network). The data can achieve the state of being “ready for use” by performing an additional step before the acquiring step. In accordance with this additional step, the data are generated in order to be acquired. The data are in particular detected or captured (for example, by an analytical device). Alternatively or additionally, the data are inputted in accordance with the additional step, for instance via interfaces. The data generated can in particular be inputted (for instance, into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. Thus, “acquiring data” can also involve commanding a device to obtain and/or provide the data to be acquired. The acquiring step in particular does not involve an invasive step which would represent a substantial physical interference with the body requiring professional medical expertise to be carried out and entailing a substantial health risk even when carried out with the required professional care and expertise. Acquiring, in particular determining, data in particular does not involve a surgical step and in particular does not involve a step of treating a human or animal body using surgery or therapy. This also applies in particular to any steps directed to determining data. In order to distinguish the different data used by the present method, the data are denoted (i.e. referred to) as “XY data” and the like and are defined by the information which they describe which is preferably called “XY information”.

A navigation system, in particular a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) in particular comprises a processor (CPU), a working memory, advantageously an indicating device for issuing an indication signal (for example, a visual indicating device such as a monitor and/or an audio indicating device such as a loudspeaker and/or a tactile indicating device such as a vibrator) and advantageously a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

The aforementioned method, in particular any method step associated with or directed to fastening the marker system, in particular the marker holder, to an anatomical body part or of introducing an induction-susceptible device into an anatomical bodypart, does not involve an invasive step which would represent the substantial physical interference with the human or animal body requiring professional medical expertise to be carried out and entailing a substantial health risk even when carried out with the required professional care and expertise. Furthermore, no part of the inventive method involves a step of treating a human or animal body using surgery or therapy.

The present invention is directed in particular to a method of determining the position of an induction-susceptible device inside an anatomical body part. This method preferably comprises a step of emitting, towards the anatomical body part and from an electromagnetic emitter device, an oscillating electromagnetic field having a predetermined first field strength. The induction-susceptible device preferably comprises a free-electron material, in particular a material comprising a metal, more particularly a ferromagnetic metal. The induction-susceptible device preferably is a medical instrument such as an electrode or another instrument (for example, a scalpel or pincers or an endoscope or suction device). According to a specific embodiment, the induction-susceptible device is an electrode for internal detection of electroencephalographic signals, in particular a strip electrode or grid electrode. The induction-susceptible device is preferably placed inside the anatomical body part, i.e. it is inserted into the patient's body (in particular, in a non-invasive manner, for example by insertion through anatomical openings such as the nose, mouth or rectum) and placed in particular at a desired location in the body. Such insertion and placement of the induction-susceptible device inside an anatomical body part is not part of the invention and in particular performed outside of the inventive method, for example before execution of the inventive method starts. The anatomical body part is in particular accessible via an anatomical opening of the patient's body so that inserting the induction-susceptible device does not require (and in particular does not comprise or encompass) any surgical activity. For example, the anatomical body part is a paranasal sinus, the stomach or part of the colon.

The electromagnetic emitter device comprises a field generating unit such as for example an oscillating circuit (LC element) which is coupled to an emission unit (for example, an antenna). The electromagnetic field generated by the field generating unit comprises, in particular consists of, wavelengths in the range of radio waves, in particular in the range of 1 mm to 1 m. The wave lengths and the first field strengths are preferably constituted such that they are enough for the electromagnetic field to penetrate at least part of the anatomical body part, in particular that part which lies in between the induction-susceptible device and the electromagnetic emitter device. Further preferably, the wave lengths and the field strength are constituted such that the electromagnetic field is able to penetrate the human skull down to a subdural region at which an internal electroencephalographic electrode is usually placed.

Preferably, an electromagnetic detector device is used to acquire an electromagnetic detection signal describing a second field strength which corresponds to the field strength of the electromagnetic field in the proximity of the anatomical body part. Preferably, the electromagnetic detector device is an electromagnetic field sensor which comprises in particular an oscillating circuit which is connected to a sensor unit such as an antenna and may therefore be activated, in particular excited, when placed in an electromagnetic field. The electromagnetic detector device is in particular placed in the proximity of the anatomical body part, more particularly on or just above its outer surface in order to determine the second field strength at that location. For example, the electromagnetic emitter device and the electromagnetic detector device are combined in one apparatus, in particular they constitute an inductive proximity switch.

The inventive method then preferably carries on with determining, based on comparing the first field strength and the second field strength, whether the second field strength differs from the first field strength. In particular, it is determined whether the second field strength is lower than the first field strength. Such a loss in field strength corresponds to a loss of energy of the electromagnetic field in the direction from the electromagnetic emitter device towards the anatomical body part. Such a loss of energy may be due to dissipation of electromagnetic field energy in the atmosphere between the electromagnetic emitter device and the outer surface of the anatomical body part and other dissipative effects. One such other dissipative effect is induction of an electric current in the induction-susceptible device which is caused by the electromagnetic field between the induction-susceptible device. The inventive method assumes that dissipation of electromagnetic field energy due to reasons other than in induction of an electric current in the induction-susceptible device can be accounted for on the basis of the skilled person's general knowledge. Such contributions to a loss of electromagnetic field energy may be added to the difference between the first field strength and the second field strength and a threshold value may be given for the remaining difference in field strength. If it is now determined that the second field strength differs from the first field strength, in particular if the remaining difference in field strength exceeds the predetermined threshold value, a corresponding position indicating signal is preferably issued froman indication device. The presence or position of the induction-susceptible device can thus be determined based on the absolute value of the difference between the first field strength and the second field strength. The position indicating signal indicates in particular the presence, more particularly also the position of the induction-susceptible device. To this end, the indication device preferably receives a signal from the electromagnetic detector device, in particular from a comparator contained in the electromagnetic detector device, which comparator performs a comparison of field strengths.

The indication device preferably is a visual indication device, in particular a light-emitting device, more particularly a light-emitting diode. The light-emitting device emits light which preferably is in at least one of the infrared wavelength range and the visible wavelength range. For example, a light-emitting diode may be electrically and operatively coupled to the electromagnetic detector device to receive an electric signal if the conditions for issuing an indicating signal are fulfilled. The light-emitting device will then start to emit light, thereby making the operator aware of the presence of the induction-susceptible device. Alternatively or additionally, the indication device may be an acoustic indication device such as a loudspeaker which reacts to the indicating signal.

In particular based on knowledge about the detection geometry of the electromagnetic detector device the position of the induction-susceptible device may be determined based on comparing the first field strength and the second field strength. In particular based on knowledge of the emission geometry of the electromagnetic emitter device, the position of the induction-susceptible device is preferably determined based on comparing the first field strength and the second field strength. If for example the relation between the first field strength and the second field strength is known in absolute terms (for example, the second field strength may be said to amount to 80% of the first field strength) the conditions for issuing a position indicating signal are fulfilled, the quantity of the first field strength and the emission direction of the electromagnetic emitter device are known, the position of the induction-susceptible device may be determined in particular in three dimensions. The position of the induction-susceptible device can be determined in particular relative to the position of the electromagnetic emitter device or relative to any other predetermined position having a known spatial relationship relative to the position of the electromagnetic emitter device.

According to a further preferable embodiment, the position of the induction-susceptible device in the anatomical body part can be determined additionally based on medical image information describing the anatomical body part. Such medical image information is for example generated by applying a medical imaging method to the anatomical body part. In the context of this disclosure, a medical imaging method is understood to encompass apparatus-based imaging methods such as conventional X-ray or X-ray-based computed tomography or ultrasound imaging.

Alternatively or additionally, at least one marker device is preferably attached with a predetermined spatial relationship to at least one of the electromagnetic emitting device and the electromagnetic detector device. The marker device is then preferably used in connection with a navigation system to track the position of the electromagnetic emitting device or the electromagnetic detector device, respectively. The indicating signal may then additionally or alternatively, be sent as a digitalized signal to the computer of the navigation system which receives the signal and, on the basis of the indicating signal and the position determined for the electromagnetic emitting device or electromagnetic detector device, respectively, the position of the induction-susceptible device in the anatomical body part can be determined (in particular by the computer of the navigation system). To this end, the anatomical body part is preferably registered with regard to a coordinate system in which the tracking by the navigation system takes place. Such a registration may be based in particular on medical image information of the anatomical body part and tracking the anatomical body part in particular by using marker devices attached to it during the execution of the inventive method. If the navigation system is used for determining the position of the induction-susceptible device, it is particularly convenient to include the indication device in the navigation system. In such a case, the indication device is embodied in particular by a monitor used for visualizing the navigation. In view of the above, the inventive method can be understood to be a method of operating a navigation system for determining the presence or position of an induction-susceptible device inside an anatomical body part which involves the aforementioned method steps and features.

The aforementioned method steps may preferably be executed for a plurality of time-sequential positions of the electromagnetic detector device relative to the anatomical body part in order to determine a trajectory of the induction-susceptible device inside the anatomical body part. For example, the electromagnetic detector device may be led by an operator along a specific path on or above the surface of the anatomical body part and a trajectory of the induction-susceptible device inside the anatomical body part is determined for each position of the electromagnetic detector device at which the conditions for issuing a position indicating signal are fulfilled.

In order to avoid a malfunction of the inventive method or system, the first field strength is preferably varied, in particular increased, during execution of the inventive method. Such an increase in particular takes place if it is determined that no electromagnetic detection signal can be acquired. Thereby, a malfunction can be avoided which is due to a first field strength which is too low to penetrate the parts of the anatomical body part lying in between the electromagnetic emitter device and the induction-susceptible device. This feature can also be employed for a measurement of the depth of the induction susceptible device inside the anatomical body part. For example, information about electromagnetic material constants of the tissue between the electromagnetic emitter device (and the electromagnetic detector device) and the induction-susceptible device is provided to the inventive method and by varying the first field strength until the conditions for issuing an indicating signal are fulfilled, the depth is determined.

According to a specific embodiment of the invention, the electromagnetic emitter device and the electromagnetic detector device can be placed in for example three directions which are preferably orthogonal to each other in order to determine the position of the induction-susceptible device in three dimensions. Alternatively or additionally, a second pair of a second electromagnetic emitter device and a second electromagnetic detection device may be simultaneously placed in preferably an orthogonal direction relative to a first pair of a first electromagnetic emitter device and a first electromagnetic detector device such that they can simultaneously detect the induction-susceptible device and support determining its position in three dimensions.

According to an even further embodiment of the invention, the electromagnetic field emitted by the electromagnetic emitter device is a modulated electromagnetic field which penetrates different depths. Based on information about the present modulation and the point in time at which the conditions for issuing a position indicating signal are fulfilled, an at least rough depth measurement of the induction-susceptible device is made possible.

Furthermore, the electromagnetic detector device may be placed at a fixed position relative to the anatomical body part and the induction-susceptible device may be moved, in particular within the anatomical body part, relative to the electromagnetic detector device. Once a position indicating signal is issued from the indication device, it is clear that the induction-susceptible device has then reached a predetermined position relative to the electromagnetic detector device.

The invention also relates to a program which, when running on a computer or when loaded onto a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

The aforementioned computer program in particular comprises code means which are adapted to cause, when the program is running on a computer or when it is loaded onto a computer, the computer to perform one or more or all of the method steps of the method as described above. Execution of the method is implemented by preferably following data processing steps.

Preferably, electromagnetic emission data comprising electromagnetic emission information is acquired. The electromagnetic emission information describes in particular an emission direction and a predetermined first field strength of the oscillating electromagnetic field to be emitted towards the anatomical body part.

Preferably, electromagnetic detection data comprising electromagnetic detection information is acquired in particular based on the electromagnetic signal describing the second field strength. In particular, the signals measured by the electromagnetic detector device are fed from the electromagnetic detector device to an analogue-digital converter and then read by the computer. The electromagnetic detection information describes in particular a second field strength.

The aforementioned comparison of the first field strength and the second field strength preferably serves as a basis for determining field strength difference data comprising field strength difference information. The field strength difference information describes in particular that the second field strength differs from the first field strength and preferably also an absolute or relative value characterising that difference.

The computer program then carries on to determine whether the field strength difference information describes that the second field strength differs from the first field strength and preferably also the (absolute or relative) amount by which it differs. Hit is determined that the field strength difference information describes that the second field strength differs from the first field strength, position indicating data is preferably determined which comprises position indicating information. The position indicating information in particular describes the presence, in particular the position of the induction-susceptible device.

In the following, an embodiment of the present invention is described with reference to a figure. This embodiment shall be understood as a mere example and not as a limitation of the invention to only those features which are shown in the figure.

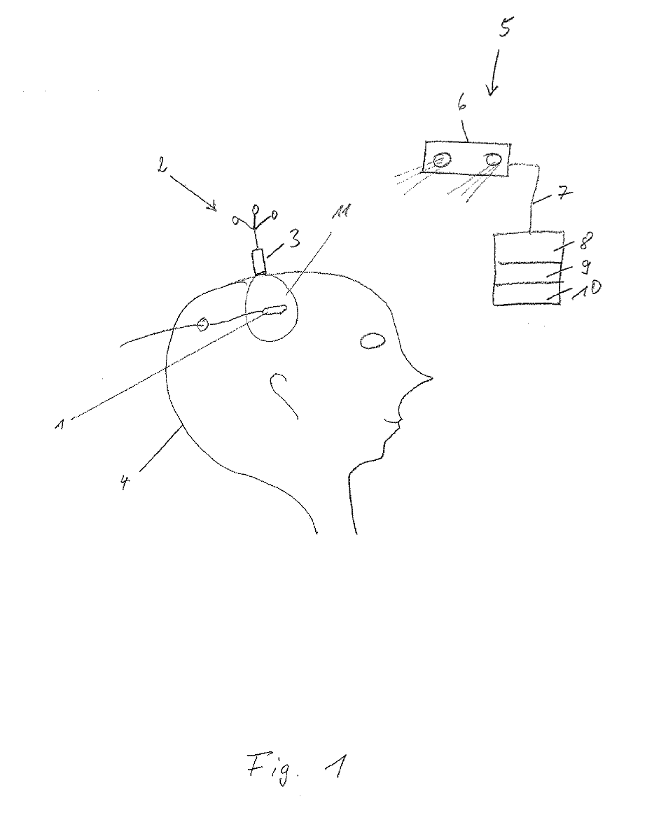

FIG. 1 shows a setup for detecting a strip electrode with a navigated inductive proximity switch.

FIG. 1 shows how to place a navigated inductive proximity switch 3 comprising the electromagnetic emitter device and the electromagnetic detector device on the surface of a patient's head representing the anatomical body part in order to detect a strip electrode 1 that is inserted into the head 4. A reference star 2 comprising three markers is fixedly attached to the inductive proximity switch 3 in order to enable tracking of the inductive proximity switch 3 by a navigation system 5. The navigation system 5 comprises a stereotactic camera 6 for optical detection of the reference star 2 and for transmitting the detection signals via a data line 7 to a computer of the navigation system 5. The computer comprises a CPU 8, a random access memory 9 and a hard disc 10 in order to process the detection signal received from the camera 6 and to convert them into positional information describing the position of the inductive proximity switch 3.

The inductive proximity switch 3 emits and detects an electromagnetic field within the proximity volume 11. If the electrode 1 enters the proximity volume 11, the position indicating signal is generated by the proximity switch 3 and in the embodiment of FIG. 1 transmitted by wireless data transmission to the computer of the navigation system 5 for further processing.

1. A method of determining the position of an induction-susceptive device inside an anatomical body part, the method comprising:
a) emitting, towards the anatomical body part and from an electromagnetic emitter device, an oscillating electromagnetic field having a predetermined first field strength;
b) acquiring, by using an electromagnetic detector device, an electromagnetic detection signal describing a second field strength which corresponds to the field strength of the electromagnetic field in the proximity of the anatomical body part;
c) determining, based on comparing the first field strength and the second field strength, whether the second field strength differs from the first field strength, and
d) if it is determined that the second field strength differs from the first field strength, issuing a corresponding position indicating signal from an indication device, which position indicating signal indicates a presence of the induction-susceptible device,
wherein at least one marker device is attached with a predetermined spatial relationship to at least one of the electromagnetic emitting device and the electromagnetic detector device and wherein the indicating signal is received, as digitized signal, by a computer of a medical navigation system used for navigating at least one of the electromagnetic emitting device and the electromagnetic detector device.

2. The method according to claim 1, wherein the induction-susceptive device comprises electrically conductive material.

3. The method according to claim 1, wherein the electromagnetic emitter device and the electromagnetic detector device are combined in one apparatus.

4. The method according to claim 1, wherein the induction-susceptible device is an electrode for internal detection of electroencephalographic signals.

5. The method according to claim 1, wherein the electromagnetic field comprises, wavelengths in the range of 1 mm to 1 m.

6. The method according to claim 1, further comprising
e) determining, based on comparing the first field strength and the second field strength, a position of the induction-susceptible device.

7. The method according claim 6, wherein it is determined that the second field strength differs from the first field strength and wherein the position of the induction-susceptible device is determined based on an absolute value of the difference between the first field strength and the second field strength.

8. The method according to claim 6, wherein the position of the induction-susceptible device is determined additionally based on medical image information describing the anatomical body part.

9. The method according to claim 1, wherein the indication device is a visual indication device, which emits light in the visible wavelength range, or wherein the indication device is an acoustic indication device.

10. (canceled)

11. The method according to claim 9, wherein the indication device is included in the medical navigation system and is embodied by a monitor used for visualizing navigation.

12. The method according to claim 1, wherein the method steps are executed for a plurality of time-sequential positions of the electromagnetic detector device relative to the anatomical body part for determining a trajectory of the induction-susceptible device inside the anatomical body part.

13. The method according to claim 1, wherein the first field strength is varied, if it is determined that no electromagnetic detection signal can be acquired.

14. A non-transitory computer-readable program storage medium storing a computer program which causes a computer of a medical navigation system, when the program is running on a processor of the computer or when it is loaded into a memory of the computer, to perform a computer-implemented method of determining the position of an induction-susceptive device inside an anatomical body part, the method comprising executing, on the processor, steps of:
a) acquiring, at the processor, electromagnetic emission data comprising electromagnetic emission information describing an emission direction and a predetermined first field strength of an oscillating electromagnetic field to be emitted from an electromagnetic emitter device towards the anatomical body part;
b) acquiring, at the processor and based on an electromagnetic signal determined using an electromagnetic detector device and describing a second field strength, electromagnetic detection data comprising electromagnetic detection information describing the second field strength;
c) determining, by the processor and based on the comparison of the first field strength and the second field strength, field strength difference data comprising field strength difference information describing whether the second field strength differs from the first field strength, and
d) if it is determined that the field strength difference information describes that the second field strength differs from the first field strength, determining, by the processor, position indicating data comprising position indicating information describing a presence of the induction-susceptible device,
wherein at least one marker device is attached with a predetermined spatial relationship to at least one of the electromagnetic emitting device and the electromagnetic detector device and wherein the position indicating information is processed, by the processor and as a digitized signal, for navigating the at least one of the electromagnetic emitting device and the electromagnetic detector device.

15. A computer comprising the program storage medium according to claim 14.

16. A medical navigation system comprising a computer, the computer having a memory and a processor, wherein the processor is configured to execute a program which causes a computer, when the program is running on the processor or when it is loaded into the memory, to perform a computer-implemented method of determining the position of an induction-susceptive device inside an anatomical body part, the method comprising executing, on the processor, steps of:
a) acquiring, at the processor, electromagnetic emission data comprising electromagnetic emission information describing an emission direction and a predetermined first field strength of an oscillating electromagnetic field to be emitted from an electromagnetic emitter device towards the anatomical body part;
b) acquiring, at the processor and based on an electromagnetic signal determined using an electromagnetic detector device and describing a second field strength, electromagnetic detection data comprising electromagnetic detection information describing the second field strength;
c) determining, by the processor and based on the comparison of the first field strength and the second field strength, field strength difference data comprising field strength difference information describing whether the second field strength differs from the first field strength, and
d) selectively determining, by the processor in accordance with a result of determining that the field strength difference information describes that the second field strength differs from the first field strength, position indicating data comprising position indicating information describing a presence of the induction-susceptible device, wherein at least one marker device is attached with a predetermined spatial relationship to at least one of the electromagnetic emitting device and the electromagnetic detector device and wherein the position indicating information is processed, by the processor and as a digitized signal, for navigating the at least one of the electromagnetic emitting device and the electromagnetic detector device.
